(19) United States
(12) Patent Application Publication
(10) Pub. No.: US 2022/0008099 A1
(43) Pub. Date: Jan. 13, 2022

(54) IMPLANTING SYSTEM FOR IMPLANTING A TOTALLY IMPLANTABLE DEVICE IN A HUMAN OR ANIMAL BODY

(71) Applicant: Medautonomic S.R.L., Venezia Mestre (IT)

(72) Inventors: Paolo FABRIS, Thiene, Vicenza (IT); Valerio CIGAINA, Cortina D'Ampezzo, Belluno (IT); Simone CIGAINA, Treviso, Villorba (IT); Arfeo CANAGLIA, Fara Vic., Vicenza (IT)

(73) Assignee: Medautonomic S.R.L., Venezia Mestre (IT)

(21) Appl. No.: 17/293,649

(22) PCT Filed: Nov. 4, 2019
(86) PCT No.: PCT/EP2019/080043

§ 371 Date: May 13, 2021

(30) Foreign Application Priority Data
Nov. 14, 2018 (IT) 102018000010318

(51) Int. Cl. A61B 17/34; A61B 1/018; A61B 1/00; A61B 1/015; A61B 5/00

(52) U.S. Cl. CPC A61B 17/3468; A61B 1/018; A61B 1/00135; A61B 2017/00907; A61B 1/015; A61B 1/00101; A61B 5/686; A61B 17/3478

(57) ABSTRACT

The implanting system (1) for implanting a totally implantable device (2) in a human or animal body comprises an introducing device (3) equipped with at least one operative channel (4a, 4b) and a transparent cover element (11) detachably attached to a distal end (6) of said introducing device (3), the cover element (11) has a rounded configuration that is outwardly convex and facilitates the sliding of said introducing device (3).

The present invention relates to an implanting system for implanting a totally implantable device in a human or animal body.

The scope extends to an implanting system for implanting any totally implantable device that can be housed in the gastric wall, inside or under the sub-mucosa or in the thickness of the wall of the entire digestive channel and/or any hollow organ or inside walls inside or outside a human or animal body.

Patent applications WO 2015/079005 and WO/2017/198532 disclose an implanting system for implanting a totally implantable device comprising an insertion device, in particular an endoscope, which has at least one operative channel in which an implanting tool is inserted.

In some cases, these implanting systems are not completely effective to allow implanting a totally implantable device under sterile conditions.

For example, in order to implant a totally implantable device in some zones, the implanting system must necessarily pass through human or animal cavities having a large number of microorganisms, leaving the totally implantable device and the implanting tool exposed to bacterial contamination.

Therefore, such implanting systems, although effective in the insertion of these devices, do not guarantee implanting without the risk of rejection by the implantation zone.

Furthermore, such implanting systems involve some difficulties in the step of introducing the insertion device, such as for example an endoscope, due to the interaction by friction of the end part of the insertion device with the tissues of the human or animal cavities that can cause bleeding and consequently reduce the level of cleanliness.

The technical task of the invention is to overcome the limits of said systems as well as to allow sterile implantation in organs which are difficult to reach.

The technical task, as well as these and other objects according to the present invention, are achieved by providing an implanting system for implanting a totally implantable device in a human or animal body comprising an introducing device equipped with at least one operative channel characterised in that it comprises a transparent cover element that is detachably attached to a distal end of said introducing device, said cover element having a rounded configuration that is outwardly convex, said cover element facilitating the sliding of said introducing device. Preferably, each operative channel has a respective distal outlet at the distal end of the introducing device, the introducing device has an external pocket for containing the totally implantable device that has a distal extraction opening and each distal outlet and distal opening are both protected by the transparent cover element.

The introducing device comprises a perforating means which is slidable along a first operative channel of the endoscope and a drawing means for drawing said totally implantable device and which is slidable along a second operative channel. The perforating means and the drawing means are protected by said transparent cover element.

The basic idea of the invention therefore consists in providing an implanting system configured to reach the implant site without exposing the totally implantable device and implanting tools to the external environment in order to overcome the criticalities linked to the difficulty of implantation without rejection.

The implanting system is thus able to convey any device in the thickness of the gastric wall, or in the thickness of the wall of the entire digestive channel or any hollow organ or cavity of the body or inside walls of the human or animal body and implant it under sterile conditions.

Preferably, the introducing device comprises a first cylindrical body having the at least one operative channel and a second tubular body that has a pocket for containing the totally implantable device and engages along the outer lateral perimeter of the first cylindrical body. Preferably the cover element is fluid sealingly attached.

The cover element has a friction coupling with the distal end of the introducing device.

The cover element is made of a medical silicone-based or PVC resin-based or a resorbable material such as crystalline cellulose.

Preferably the perforating means comprises an axially hollow needle connected to a flexible feed tube for feeding a fluid, an axially hollow housing body along which the needle is slidably extractable and a flexible sheath, solidly joined to the housing body, wherein said flexible tube is slidable.

The flexible sheath has a curved configuration at rest.

Preferably the drawing means of the device comprises a clamp having a first and a second fastening element translatable relative to one another between a fastening position and a release position in opposition and through the action of said elastic element.

The elastic element preferably comprises a spiral spring that has a first end fixed to the first fastening element and a second end fixed to the second fastening element and in the fastening position the spiral spring is tensioned by traction.

The second fastening element is formed by a longitudinal probe slidably positioned inside the spiral spring.

Preferably the insertion device is an endoscope.

Preferably the totally implantable device has a longitudinal piezoelectric element that is retained by the clamp.

The positioning of the device inside a biological tissue comprises in sequence the steps of:
attaching the device contained in the pocket to said drawing means;
covering the opening of the pocket, the perforating means and the drawing means with the cover element, fixing it to the distal end of the insertion device,
reaching a predetermined implant tissue zone with said distal end of the insertion device,
guiding said perforating means outside the distal outlet of the operative channel where they are housed.
releasing the cover element under the pushing action of the perforating means,
extracting said needle from said housing body and creating an implant site by feeding said needle with a quantity of fluid that is injected into said tissue zone so as to create a localised tissue swelling that acts as the implant chamber,
making the perforating means penetrate in the implant chamber thus created;
retracting the needle inside the housing body and making the perforating means further penetrate beneath the tissue in order to facilitate the detachment of the underlying surface;
repeating this operation as many times as necessary to create an implant chamber of adequate size to accommodate the device to be implanted,
retracting the perforating means from the tissue swelling that acts as an implant chamber,
extracting the drawing means from the operative channel housing it,
inserting the drawing means inside the tissue swelling that acts as an implant chamber before the perforating means is completely retracted from the swelling,
continuing the penetration up to drawing the device inside the implant chamber,
before the totally implantable device is completely inside the implant chamber, fully retracting the perforating means,
once the totally implantable device is completely inside the implant site, releasing the totally implantable device from the clamp and selectively retracting the drawing means, leaving the totally implantable device in the implant chamber.

The fluid used to create the implant site can be distilled water or a physiological solution or a medicated solution.

Further features and advantages of the invention will more fully emerge from the description of a preferred but non-exclusive embodiment of the implanting tool according to the invention, illustrated by way of an indicative and non-limiting example in the appended drawings, in which:

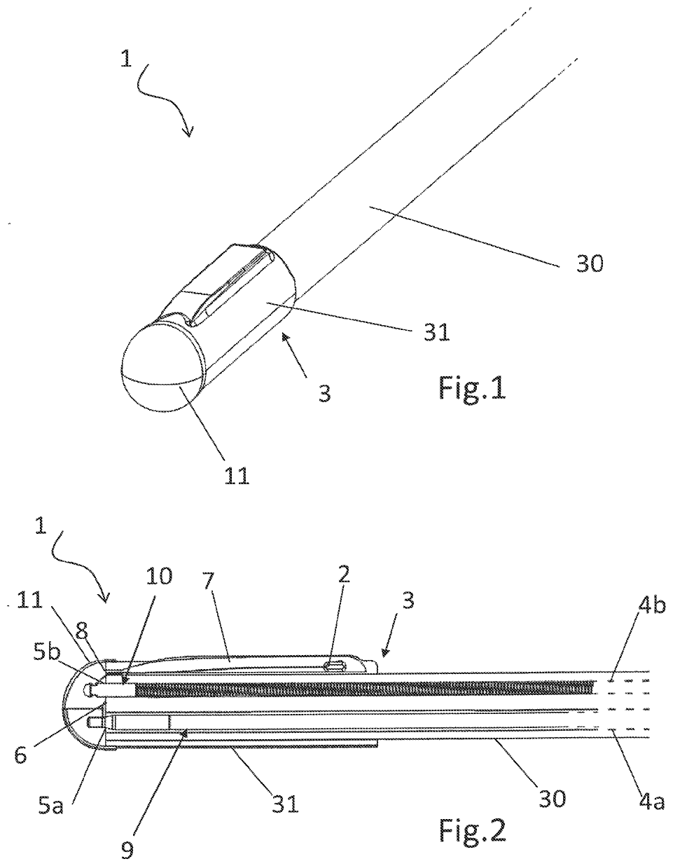

FIG. 1 shows a perspective view of the implanting system;

FIG. 2 shows the implanting system of FIG. 1 in a sectional view with respect to an axial plane;

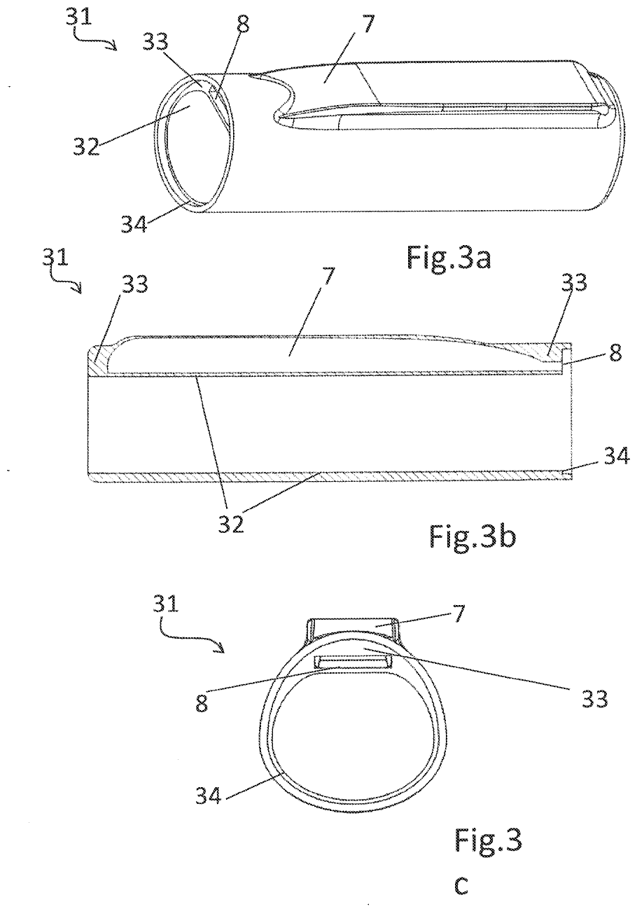

FIGS. 3a-3c respectively show a perspective view, a sectional view and a front view of the second tubular body of the endoscope;

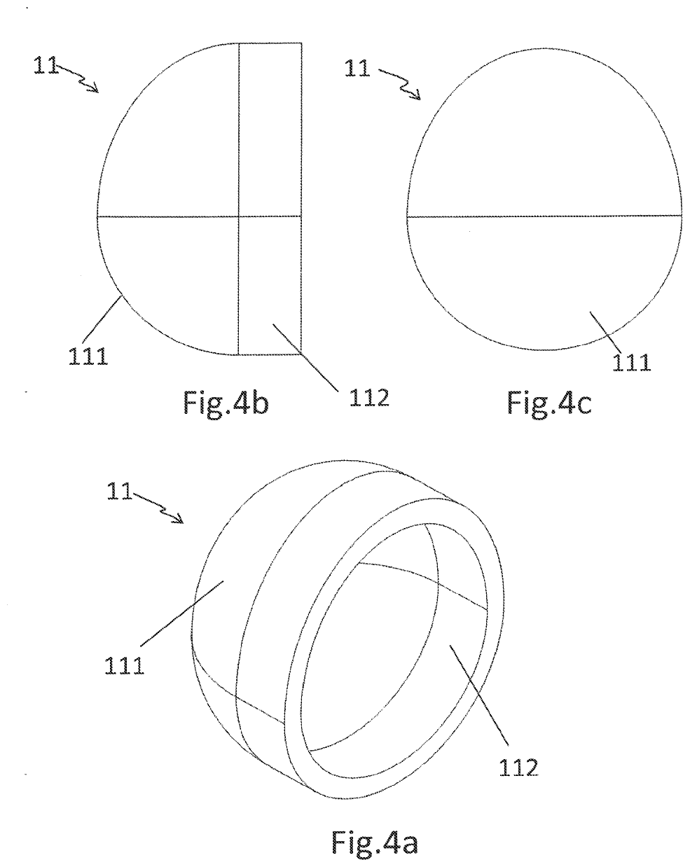

FIGS. 4a-4c show a perspective view, a side view and a front view of the cover element of the implanting system of FIG. 1;

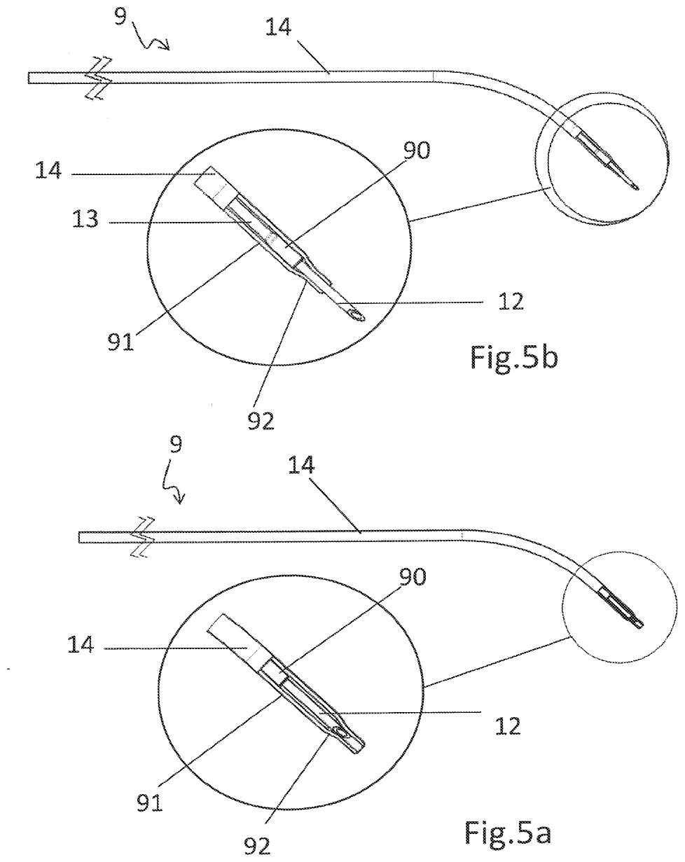

FIGS. 5a-5b show a schematic view of the perforating means with the detail of the needle in the extracted and the retracted position;

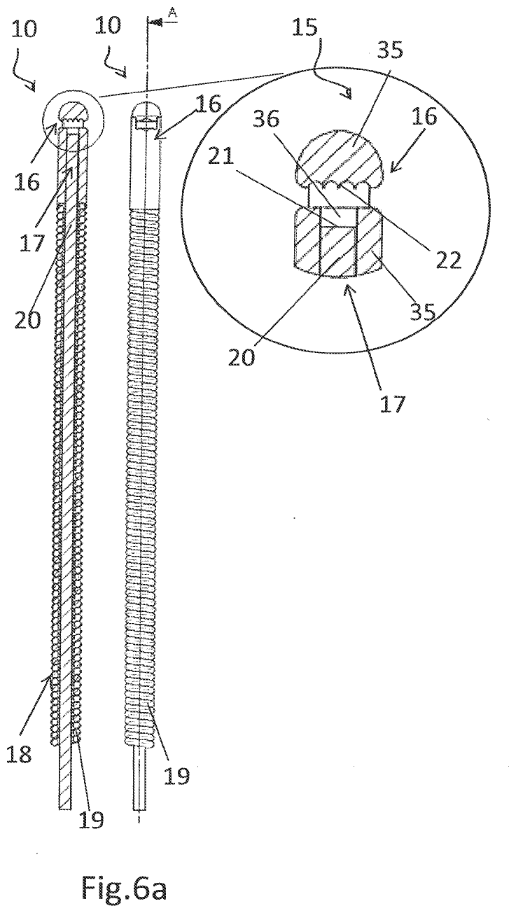

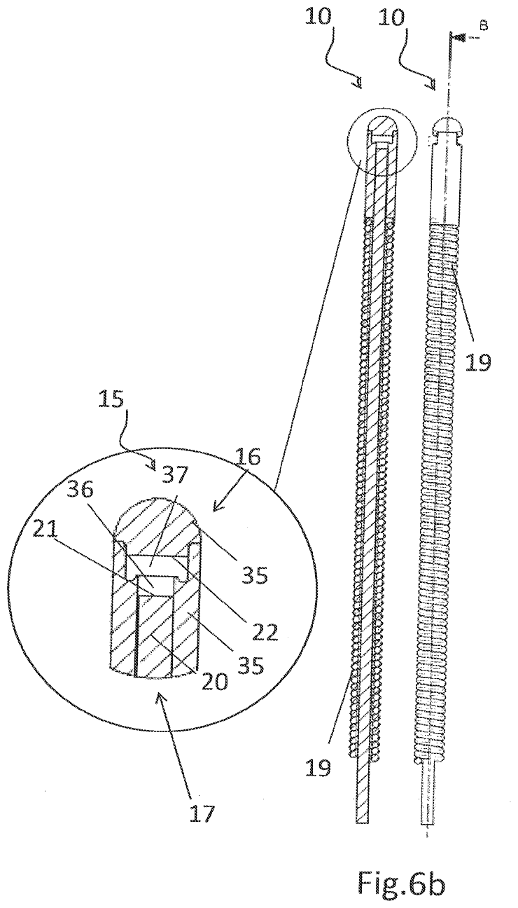

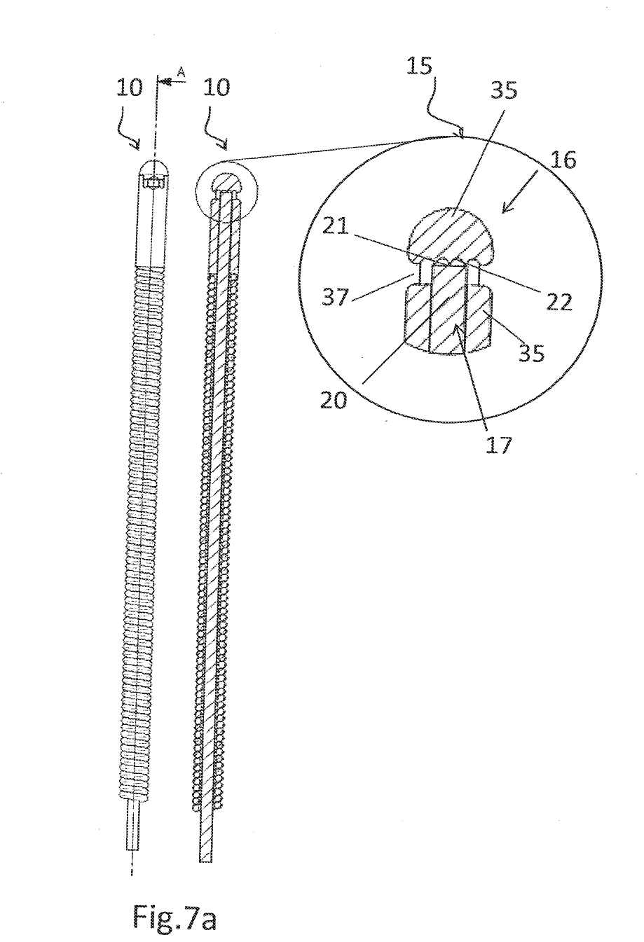

FIGS. 6a-6b respectively show a side view, a sectional view and a detail of the clamp of the drawing means with the clamp in the release position;

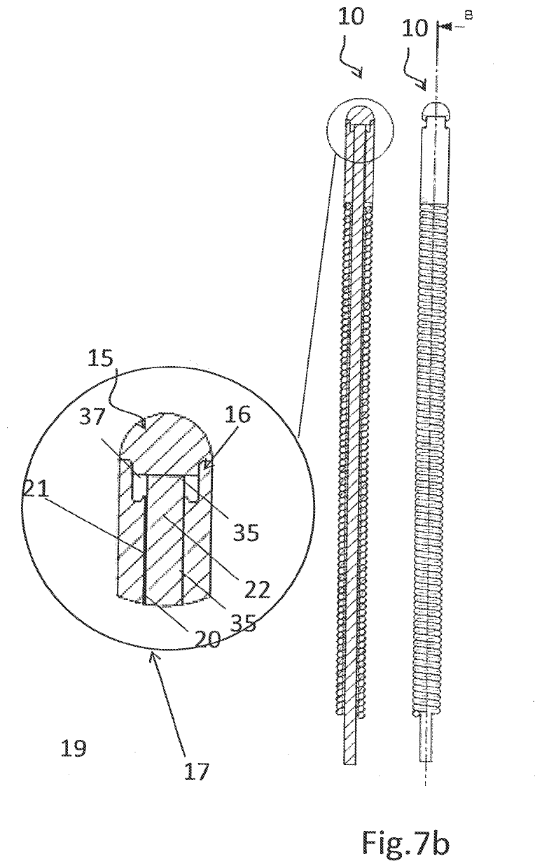

FIGS. 7a-7b respectively show a side view, a sectional view and a detail of the clamp of the drawing means with the clamp in the fastening position;

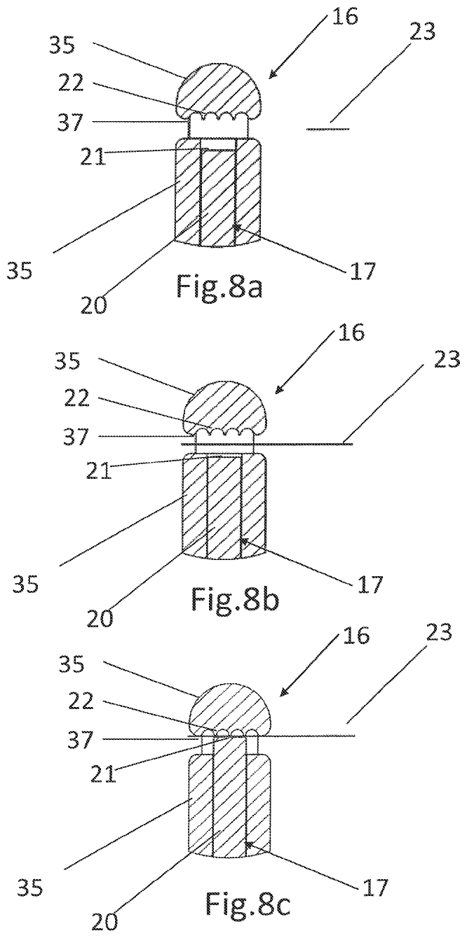

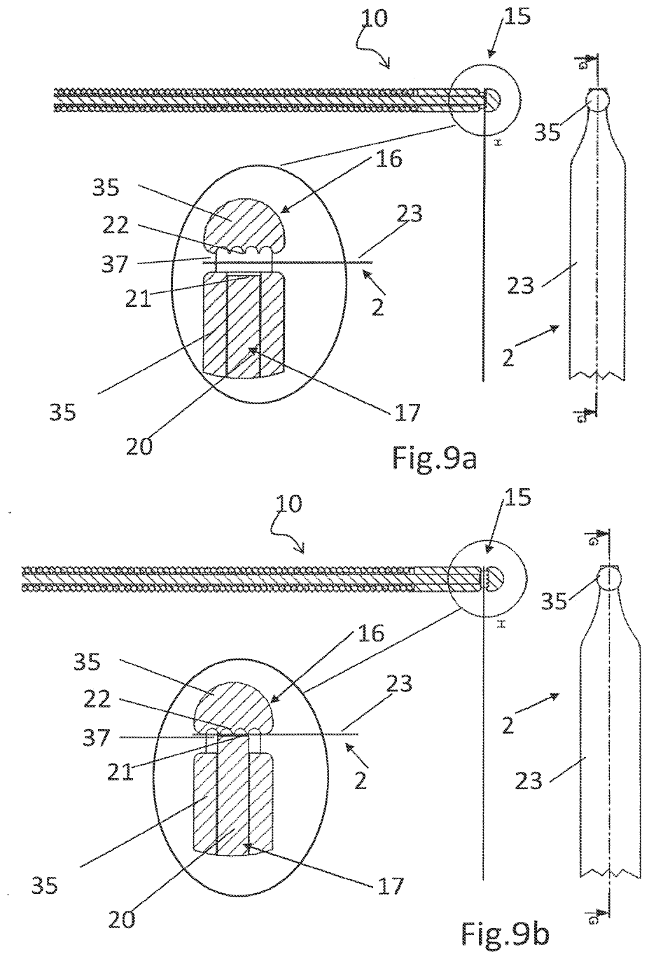

FIGS. 8a-8c and 9a-9b show sectional views, side views and upper views of the attaching of the device to the drawing means of the implanting system of FIG. 2;

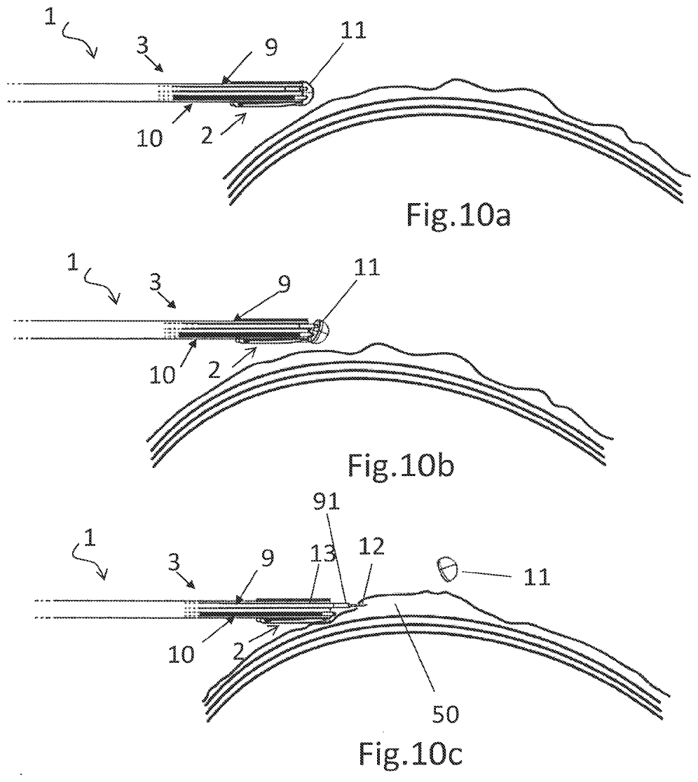

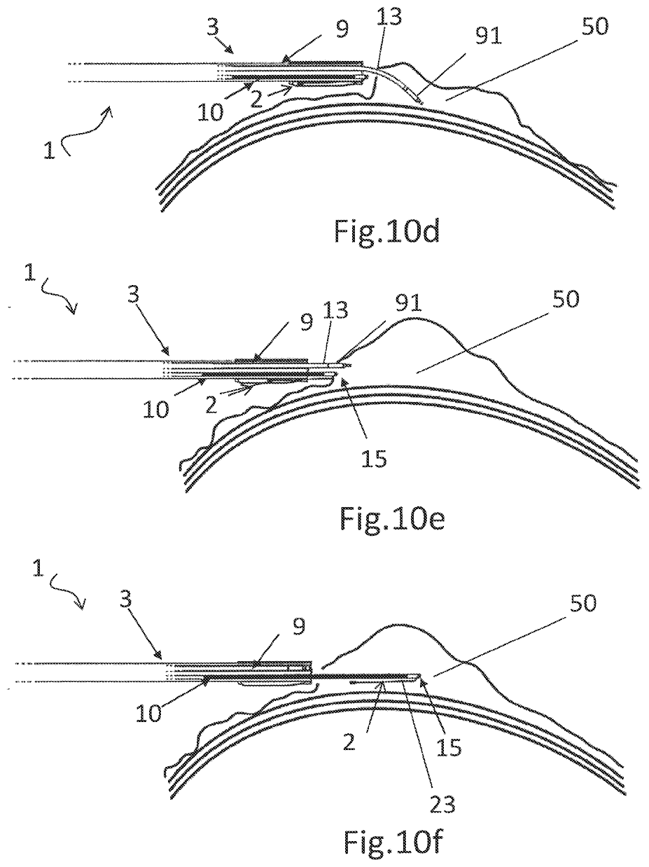

FIGS. 10a-10g show schematic views of the implanting sequence of the device using the implanting system of FIG. 1;

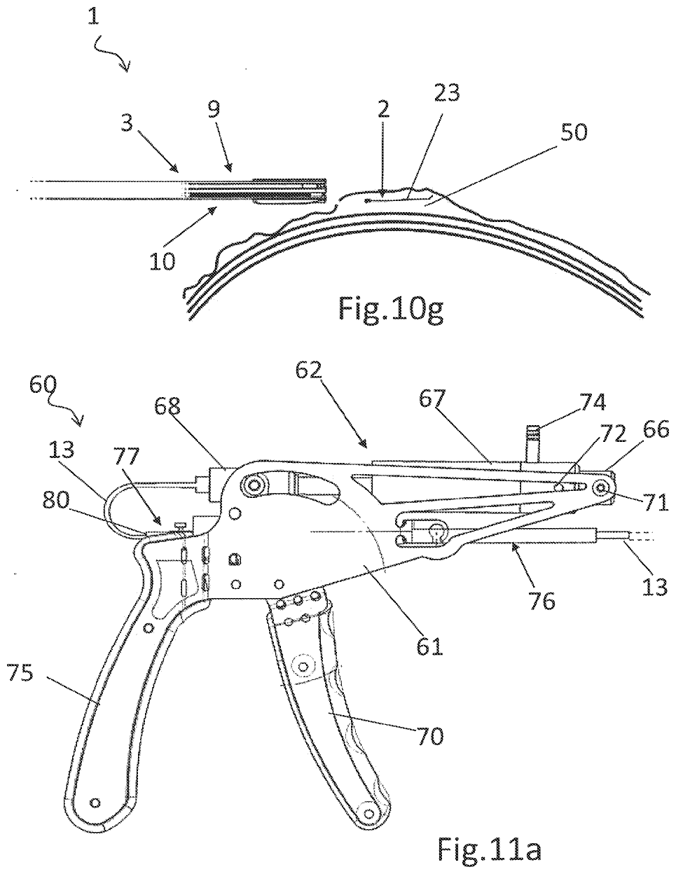

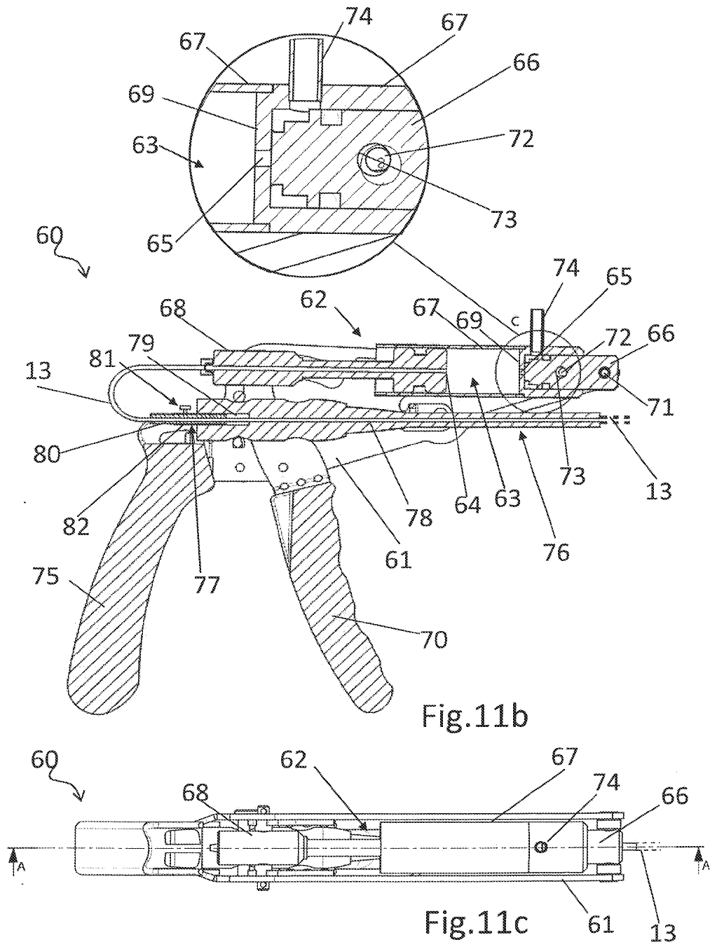

FIGS. 11a-11c show a side view, a top view and a sectional side view of a dosing unit of the fluid.

The implanting system 1 for implanting a totally implantable device 2 in a human or animal body comprises an introducing device 3 provided with at least one operative channel 4a, 4b. The introducing device 3 comprises a proximal end on the operator side and a distal end 6 on the opposite side.

In the description that follows the term distal and proximal are also defined with respect to the position of the operator.

The introducing device 3 comprises a transparent cover element 11 detachably attached to the distal end 6.

The cover element 11 has a rounded configuration that is outwardly convex to facilitate the sliding of the distal end 6 of the introducing device 3 to reach a predetermined implant tissue zone.

Each operative channel 4a, 4b of the introducing device 3 has a respective distal outlet 5a 5b at the distal end 6 of the introducing device 3.

The introducing device 3 comprises an external pocket 7 for containing the device 2.

The external pocket 7 has a distal extraction opening 8 for extracting the totally implantable device 2.

In particular, the distal opening 8 is facing the same direction in which each distal outlet 5a, 5b of each operative channel 4a, 4b of the introducing device 3 is facing.

The distal opening 8 is preferably located at the distal end 6 of the introducing device 3.

Each distal outlet 5a, 5b and the distal opening 8 are protected by the cover element 11.

In greater detail, the introducing device 3 comprises a first cylindrical body 30 which is internally provided with said at least one operative channel 4a, 4b and a second tubular body 31, preferably structurally separated from said first cylindrical body 30, which has said pocket 7 for containing the totally implantable device 2.

In particular, the second tubular body 31 comprises a cylindrical inner lateral surface 32 matching the shape of the outer lateral surface of the first cylindrical body 30 and a longitudinal zone 33 of increased thickness where the pocket 7 for containing the device 2 is formed.

The second tubular body 31 can comprise at its distal end a perimeter shoulder 34 defined by a discontinuity in the thickness of the second tubular body 31 itself.

The opening 8 of the pocket 7 is formed at the distal end of the second tubular body 31 or, if present, of the perimeter shoulder 34 of the second tubular body 31.

It should be noted that the second tubular body 31 is fitted on the outer surface of the first cylindrical body 30, and is in particular engaged along the outer perimeter of the first cylindrical body 30 so that its distal end is aligned with each distal outlet 5a, 5b of each operative channel 4a, 4b.

Preferably, the implanting system 1 comprises a perforating means 9 which is slidable along a first channel 4a of the introducing device 3 and a drawing means 10 of the device 2 which is slidable along a second operative channel 4b.

In particular, the perforating means 9 is configured to create a tissue swelling 50 in which the totally implantable device 2 can be introduced, while the drawing means 10 is configured to draw and release said device 2 inside this tissue swelling 50.

It should be noted that said perforating means 9 and said drawing means 10 are slidable along the corresponding operative channels 4a, 4b and extend beyond the respective distal outlets 5a, 5b. Advantageously, the perforating means 9 and the drawing means 10 are protected by the cover element 11.

In this way, the implanting system 1 can be used to reach the implant tissue zones in which the sterility of the totally implantable device 2 and instruments positioned inside the operative channels 4a, 4b of the introducing device 3 would have been compromised to obtain an implantation without rejection.

The cover element 11, being releasable from the distal end 6 of the introducing device 3, covers the implanting tools—the perforating means 9 and the drawing means 10- and the totally implantable device 2 until the vicinity of the implant tissue zone is reached, in which the cover element 11 is releases released and the implanting takes place by using still-sterile implanting tools and totally implantable device 2.

Advantageously, the cover element 11 is fluid sealingly attached to the introducing device 3 in such a way that dirt and bacterial agents present in the environment outside the introducing device 3 cannot come into contact with the implanting tools—the perforating means 9 and the drawing means 10- and the totally implantable device 2.

Advantageously, the cover element 11 has a friction coupling with the distal end 6 of the introducing device 3.

For example, the cover element 11 is constrained by pressure on the distal end of the second tubular body 31.

In particular, the cover element 11 comprises a dome 111 having a base with an oval profile and a collar 112 that extends from the oval base.

It should be noted that the collar 112 of the cover element 11 attaches to the perimeter surface of the distal end 6 of the introducing device 3.

In particular, the collar 112 can engage the inner surface of the distal end of the second tubular body 31 or preferably as shown in FIG. 2, on the outer surface of the tubular body 31.

In practice, the cover element 11 is fitted on the outer lateral surface of the second tubular body 31 of the introducing device 3.

In this way, the cover element 11 engages the introducing device 3 by friction and creates a fluidly sealed containment chamber that protects the perforating means 9, the drawing means 10 and the totally implantable device 2.

In particular, the cover element 11 can be made of PVC resin-based or medical silicone or resorbable material such as crystalline cellulose.

It should be noted that in order to obtain the perfect consistency of the cover element 11, use is made of a silicone-based material, or equivalent, with a hardness comprised between 1 shore and 50 shore, more preferably comprised between 10 shore and 35 shore, even more preferably between 10 shore and 12 shore.

In reaching a predetermined implant tissue zone, the cover element 11 acts as a buffer between the distal end 6 of the introducing device 3 and the tissue walls and slides on the tissue walls without damaging them.

The perforating means 9 of the implanting system 2 comprises an axially hollow needle 12 connected to a flexible feed tube 13 for feeding a fluid through a fitting 90.

Preferably the needle 12 is floating and can be moved by a control system, described below, which acts on the flexible feed tube 13 from the proximal end of the introducing device 3.

The perforating means 9 comprises an axially hollow housing body 91 along which the needle is slidably extractable and a flexible sheath 14 solidly joined to the housing body 91 in which the flexible hose 13 is slidable.

This housing body 91 has a tapered front part 92 with a blunt tip comprising an inner blocking surface against which the fitting 90 abuts.

As can be seen in FIGS. 5a and 5b, the stroke of the needle 12 in the housing body 91 is delimited between an extracted position in which the fitting 90 abuts against the inner locking surface of the housing body 91 and a retracted position in which the fitting 90 abuts against the distal end of the flexible sheath 14.

The flexible sheath 14 can have a curved configuration at rest, as can be seen in FIGS. 5a and 5b.

Substantially when the perforating means 9 is retracted into its operative channel 4a of the introducing device 3, the flexible sheath 14 is forced into an upright position by the channel 4 itself.

Instead, when the perforating means 9 is in the extracted position with respect to the distal opening 5a of the operative channel 4a, the flexible sheath 14 can assume the curved configuration that allows, in the case of implanting in curved tissue zones, following the profile of the implanting zone and facilitating the perforation and creation of the implant chamber.

The implanting system 1 comprises drawing means 10 for drawing and positioning the totally implantable device 2 in the tissue swelling 50 made through the introduction of the perforating means 9 in the implant tissue zone.

The drawing means 10 comprises a clamp 15 which has a first and a second fastening element 16, 17 connected by an elastic element 18.

The fastening elements 16, 17 are translatable relative to one another between a fastening position and a release position in opposition and through the action of the elastic element 18.

In a preferred embodiment of the invention, the elastic element 18 comprises a spiral spring 19. The first fastening element 16 is fixed to the distal end of the spiral spring 19, while the second fastening element 17 is fixed to the proximal end of the spiral spring 19.

Advantageously, in the fastening position, the spiral spring 19 is tensioned by traction so that the intervention of the operator is only required for moving the clamp 15 towards the release position.

In a preferred embodiment, the first fastening element 16 comprises a cylindrical body 35 fixed to the distal end of the spiral spring 19.

The cylindrical body 35 has an axial cavity 36, a closed distal end, an open proximal end, and a transverse through-opening 37 for access to said axial cavity 36.

Advantageously, the closed distal end has a toothed inner surface 22.

In a preferred embodiment, the second fastening element 17 comprises a longitudinal probe 20 slidably positioned inside the spiral spring 19 and inside said cavity 36 of the cylindrical body 35.

The longitudinal probe 20 has a distal end 21 which in the fastening position of the clamp 15 is in abutment with the toothed surface 22 of the cylindrical body 35.

The longitudinal probe 20 has a length greater than the sum of the length of the spiral spring 19 under tension, in other words the length of the spiral spring 19 when the clamp 15 is in the fastening position, and the length of the cylindrical body 35.

The longitudinal probe 20 definitively has a first portion including its proximal end which extends outside of the proximal end of the spiral spring 19 fixed to the longitudinal probe 20 and a second portion including its distal end 21 which extends outside of the distal end of the spiral spring 19 and slides inside the axial cavity 36 of the cylindrical body 35.

The proximal end of the spiral spring 19 and the proximal end of the longitudinal probe 20 protrude from the proximal end of the introducing device 3 in order to be able to be manipulated by the operator.

The totally implantable device 2 is configured in such a way as to be attached by the clamp 15 in order to be implanted.

Preferably, the totally implantable device 2 comprises a possibly programmable electroneuromodulation and/or stimulation and/or diagnostic data recording module, an electrical energy accumulator necessary for its operation and a longitudinal piezoelectric element 23 for generating electric pulses for the recovery of electrical energy from the natural movement of the tissue inside which the totally implantable device 2 is implanted.

The operation of the clamp 15 is briefly as follows.

Initially the clamp 15 is automatically in the fastening position due to the action of the spiral spring 19 which retains the first fastening element 16 against the second fastening element 17.

To open the clamp 15, the operator grasps the longitudinal probe 20 with one hand at a point projecting from the proximal end of the endoscope 3 and grasps the spiral spring 19 with the other hand at a point projecting from the proximal end of the endoscope 3 and pushes it in the proximal-distal direction, deforming it so as to move the distal end 21 of the longitudinal probe 20 away from the toothed inner surface 22 of the cylindrical body 35.

With the clamp 15 in the release position, the longitudinal piezoelectric element 23 of the device 2 is inserted in the transverse opening 37 of the cylindrical body 35 as schematically shown in FIGS. 8a, 8b and 9a.

Once the longitudinal piezoelectric element 23 is inserted in the device 2, the operator releases the spiral spring 19 and the clamp 15 automatically moves to the fastening position in which the longitudinal piezoelectric element 23 is retained by the clamp 15, in particular between the distal end 21 of the longitudinal probe 20 and the toothed surface 22 of the cylindrical body 35, as shown in FIGS. 8c and 9b.

The implanting system 1 for implanting a totally implantable device in a human or animal body comprises a dosing unit 60 for dosing said fluid to which the flexible feed tube 13 for feeding the fluid to the perforating means 9 is connected, as shown in FIGS. 11a-11c.

The dosing unit 60 for dosing the fluid has a frame 61 that supports the pumping means 62 of the fluid.

The fluid pumping means 62 comprises a dosing chamber 63 that has a permanently open outlet route 64 and an inlet route 65 that can be opened and closed by a shutter 66 fixed to the frame 61.

The dosing chamber 63 is delimited by a cylindrical wall 67 that is movable relative to the shutter 66 and by a piston 68 which is slidable, with friction, along said cylindrical wall 67.

In particular, the cylindrical wall 67 has a transverse partition 69 inside which in turn comprises said inlet route 65 in the form of a through hole.

The piston 68 is actuatable by a manual lever 70 pivoted to the frame 61 and has said outlet route 64 in the form of at least one axial through-hole.

The outlet route 64 of the dosing chamber 63 is connected to the flexible feed tube 13 for feeding the fluid through a fitting.

The inlet route 65 can be automatically opened and closed by the movement of the cylindrical wall 67 relative to the fixed shutter 66 due to the friction coupling between the cylindrical wall 67 and the piston 68 which is slidable inside the dosing chamber 63.

The shutter 66 is partially inserted inside the cylindrical wall 67 on the part opposite the dosing chamber 63 relative to the transverse partition 69.

In greater detail, the shutter 66 has a friction coupling with the cylindrical wall 67 and is fixed to the frame 61 through a first pivot 71.

The cylindrical wall 67 is connected to the shutter 66 through a second pivot 72 housed in a seat 73 present on the shutter 66.

In particular, this seat 73 has larger dimensions relative to the second pivot 72, thus allowing the second pivot 72 and consequently the cylindrical wall 67 a longitudinal stroke relative to the shutter 66 between a closed position and an open position of the inlet route 65 of the dosing chamber 63, in which respectively the shutter 66 is in abutment with the transverse partition 69 and the shutter 66 is distanced from the transverse partition 69.

It should be noted that the cylindrical wall 67, on the part in which the shutter 66 is inserted, has a duct 74 for connection to a source of said fluid. In particular, this duct 74 is in fluid communication with the dosing chamber 63 when the inlet route 65 is in the open position, in other words when the shutter 66 is distanced from the transverse partition 69 of the cylindrical wall 67.

Preferably, the frame 61 comprises a handle 75 that allows an operator to easily operate the manual lever 70 for the actuation of the piston 68 so as to fill and empty fluid in the dosing chamber 63.

The operation of the pumping means 62 for feeding measurable amounts of fluid to the flexible tube 13 is now described.

Initially, the piston 68 is fully inserted in the dosing chamber 63, in other words it is in abutment with the transverse partition 69 on the opposite side from the shutter 66, and the inlet route 65 of the dosing chamber 63 is in the closed position, in other words the shutter 66 is in abutment with the transverse partition 69 on the part opposite the dosing chamber 63.

By operating the manual lever 70, an operator causes the piston 68 to retract relative to the cylindrical wall 67 and the piston 68 retracts the cylindrical wall 67 by friction with respect to the shutter 66, distancing the transverse partition 69 from the shutter 66 to place the inlet route 65 of the dosing chamber 63 in the open position.

Further retracting the piston 68 in the dosing chamber 63 a vacuum is created which draws the inlet fluid from the connection duct 74 to the source of the fluid.

When the piston 68 is at its endstroke, the dosing chamber 63 is completely filled with fluid.

By operating the manual lever 70, an operator causes the piston 68 to advance inside the dosing chamber 63 and by friction the piston 68 makes the cylindrical wall 67 approach, as well as the transverse partition 69, the fixed shutter 66, closing the inlet route 65 of the dosing chamber 63. Causing the further advancement of the piston 68 inside the dosing chamber 63, the fluid is put under pressure and causes the fluid to flow inside the outlet route 64 and inside the flexible feed tube 13.

By repeating the loading operation of the dosing chamber 63, thanks to the capillary forces and to the length of the flexible tube 13 normally used in examinations or endoscopic operations, the fluid already present in the outlet route 64 and in the flexible tube 13 does not return to the dosing chamber 63.

In this way, the dosing unit 60 of the fluid allows an operator to pump measurable amounts of fluid without any limits to quantity.

It should be noted that the dosing unit 60 of the fluid comprises a longitudinal sliding guide 76 for the flexible tube 13 and an adjustment system 77 for adjusting the position of the tube 13 inside the longitudinal guide 76.

The longitudinal guide 76 is constrained to the frame 61 and has a channel 78 of a size matching the size of the flexible tube 13 and a locking seat 79, connected to said channel 78, of a size greater than the size of the flexible tube 13.

The adjustment system 77 comprises a sleeve 80 solidly joined to a portion of the flexible tube 13 and insertable in said locking seat 79, and locking means 81 of the position of the sleeve 80 with respect to the locking seat 79 of the longitudinal guide 76.

In particular, the locking means 81 comprises a screw lock realised on an extension 82 of the locking seat 79 projecting from the longitudinal guide 76.

Preferably, the sleeve 80 has a size matching the locking seat 79 and its extension 82.

Substantially, the sleeve 80 can slide between a position of maximum retraction relative to the locking seat 79 to a position of complete insertion in the locking seat 79.

By changing the position of the sleeve 80, an operator accordingly makes the flexible tube 13 slide inside the longitudinal guide 76 and actuates the movements of the needle 12 by extracting and retracting it relative to the housing body 91.

The introducing device 3 can be any device for conveying a totally implantable device 2 inside a human or animal body.

Advantageously, the introducing device 3 can also be an endoscope.

The following description is given with reference to implanting the totally implantable device 2 in the intragastric wall, so-called sub-mucosa, using a gastroscope as the introducing device 3. A similar approach can be used for implanting in other body seats (human or animal).

The needle 12, in the arrangement shown in FIG. 5b, and therefore completely exposed, thanks to its conformation and arrangement, can be brought into contact with the wall in which the implanting takes place, in the case used as an example the gastric mucosa, is able to penetrate the mucosa by a few millimetres, and allows the injection of the fluid adapted, prevalently but not exclusively, to create the tissue swelling 50, or implant chamber, or sufficient space for the subsequent insertion of the totally implantable device 2.

The perforating means 9 allows the penetration in the tissues thanks to the tapered front part 92 with a blunt tip of the housing body 91.

After the creation of the swelling 50 and a sufficient penetration of the perforating means 9 inside the tissues, the perforating means 9 is retracted up to reaching the entrance of the implant chamber.

Before the perforating means 9 is extracted completely from the entrance to the implant chamber, the drawing means 10 which draws the totally implantable device 2 until it reaches the entrance is made to advance, where it is pushed inside the tissues thanks to the thrust exerted by the longitudinal probe 20 of the second fastening element 17, while the perforating means 9 is extracted.

With particular reference to FIGS. 10a-10g, the procedure for using the implanting system 1 in the intragastric wall, so-called sub-mucosa, is briefly as follows.

The gastroscope 3 of the implanting system 1 is brought near the tissue where the insertion will take place, with the totally implantable device 2, perforating means 9 and drawing means 10 completely protected by the cover element 11, as shown in FIG. 10a.

The perforating means 9 is pushed outside its operative channel 4a, keeping the needle 12 in the retracted position, until it comes into contact with the cover element 11.

As can be seen in FIG. 10b, by further pushing the perforating means 11 against the cover element 11, the latter disengages from the distal end 6 of the gastroscope.

Once the cover element 11 is released, the needle 12 is put in the extracted position and inserts in the soft tissue, for example below the gastric mucosa as shown in FIG. 10c.

Fluid is injected through the flexible tube 13, with which the tissue swelling 50, or implant chamber, is created in the soft tissue, for example below the gastric mucosa.

After the injection of the fluid, the needle 12 retracts, and with the needle 12 in the retracted position, the housing body 91 of the needle 12 is pushed further inside the tissue swelling 50 to dissect the tissue. As shown in FIG. 10d, the curved portion of the flexible sheath 14 of the perforating means 9 allows the tapered front part 92 with a blunt tip of the housing body 90 to dissect the tissue along its curvature.

The perforating means 9 is retracted until it reaches the entrance of the tissue swelling 50 and before removing it completely, the drawing means 10 is pushed from the operative channel 4b of the gastroscope 3 inside the tissue swelling 50, as shown in FIG. 10e. In this manner, the insertion of the drawing means 10 is facilitated by the partial extraction of the perforating means 9, keeping the entrance of the tissue swelling 50 open and guiding the drawing means 10 inside. Once the drawing means 10 has been inserted, the perforating means 9 is completely retracted and, as shown in FIG. 10f, the drawing means is pushed up to the implantation site inside the tissue swelling 50 up to the insertion of the totally implantable device 2.

At this point the clamp 15 for depositing the totally implantable device 2 in the soft tissue is opened.

Finally, the drawing means 10 is withdrawn, leaving the totally implantable device 2 deposited in the appropriate seat as shown in FIG. 10g.

Once the implantation is concluded, the totally implantable device 2 lies entirely within the tissues, more specifically, below the stomach mucosa.

The implanting system 1 as conceived herein is susceptible to numerous modifications and variants, all falling within the scope of the inventive concept; furthermore, all the details are replaceable by technically equivalent elements. In practice, the materials used, as well as the dimensions, can be any according to the needs and the state of the art.

1. An implanting system (1) for implanting a totally implantable device (2) in a human or animal body comprising an endoscope (3) equipped with at least one operative channel (4a, 4b), characterised in that it comprises a transparent cover element (11) that is detachably attached to a distal end (6) of said endoscope (3), said cover element (11) having a rounded configuration that is outwardly convex, said cover element (11) facilitating the sliding of said endoscope (3), each operative channel (4a, 4b) having a respective distal outlet (5a, 5b) at said distal end (6) of said endoscope (3), said endoscope (3) having an external pocket (7) for containing said totally implantable device (2), said pocket (7) having a distal extraction opening (8) for extracting said totally implantable device (2), each distal outlet (5a, 5b) and said distal extraction opening (8) being covered and protected by said cover element (11).

2. The implanting system (1) for implanting a totally implantable device (2) in a human or animal body according to claim 1, characterised in that said endoscope (3) has a first cylindrical body (30) having each operative channel (4a, 4b) and a second tubular body (31), said second tubular body (31) having said pocket (7) and engaging along the outer lateral perimeter of said first cylindrical body (30).

3. The implanting system (1) for implanting a totally implantable device (2) in a human or animal body according to claim 1, characterised in that said cover element (11) is fluid sealingly attached.

4. The implanting system (1) for implanting a totally implantable device (2) in a human or animal body according to claim 1, characterised in that said cover element (11) has a friction coupling with said distal end (6).

5. The implanting system (1) for implanting a totally implantable device (2) in a human or animal body according to claim 1, characterised in that said cover element (11) is made of a medical silicone-based or PVC resin-based material or a resorbable material such as crystalline cellulose.

6. The implanting system (1) for implanting a totally implantable device (2) in a human or animal body according to claim 1, characterised in that said endoscope (3) comprises a perforating means (9) which is slidable along a first operative channel (4a) of said endoscope (3), a drawing means (10) for drawing said totally implantable device (2) and which is slidable along a second operative channel (4b) of said endoscope (3), said perforating means (9) and said drawing means (10) being protected by said cover element (11).

7. The implanting system (1) for implanting a totally implantable device (2) in a human or animal body according to claim 1, characterised in that said perforating means (9) comprises an axially hollow needle (12) connected to a flexible feed tube (13) for feeding a fluid, an axially hollow housing body (91) along which said needle (12) is slidably extractable, and a flexible sheath (14) solidly joined to said housing body (91), said flexible tube (13) being slidable in said flexible sheath (14).

8. The implanting system (1) for implanting a totally implantable device (2) in a human or animal body according to claim 1, characterised in that said flexible sheath (14) has a curved configuration at rest.

9. The implanting system (1) for implanting a totally implantable device (2) in a human or animal body according to claim 7, characterised in that it comprises a dosing unit (60) for dosing said fluid having a frame (61) supporting a fluid pumping means (62) comprising a dosing chamber (63) having an inlet route (65) provided with a shutter (66) fixed to said frame (61) and a permanently open outlet route (64), said dosing chamber (63) being delimited by a cylindrical wall (67) that is movable relative to the shutter (66) and by a piston (68) that is slidable, with friction, along said cylindrical wall (67) and actuatable by a manual lever (70) pivoted to said frame (61), said piston (68) having said outlet route (64) in the form of at least one axial through-hole, said inlet route (65) being able to be automatically opened and closed by the movement of the cylindrical wall (67) relative to the shutter (66) due to the friction coupling between the cylindrical wall (67) and the piston (68), said outlet route (64) being connected to said flexible tube (13) for feeding the fluid.

10. The implanting system (1) for implanting a totally implantable device (2) in a human or animal body according to claim 9, characterised in that said dosing unit (60) for dosing said fluid comprises a longitudinal sliding guide (76) for the flexible tube (13) and an adjustment system (77) for adjusting the position of said flexible tube inside the longitudinal guide (76).

11. The implanting system (1) for implanting a totally implantable device (2) in a human or animal body according to claim 6, characterised in that said drawing means (10) comprises a clamp (15) having a first and a second fastening element (16, 17), said first and said second fastening element (16, 17) being translatable relative to one another between a fastening position and a release position in opposition and through the action of an elastic element (18).

12. The implanting system (1) for implanting a totally implantable device (2) in a human or animal body according to claim 11, characterised in that said elastic element (18) comprises a spiral spring (19) having a first end fixed to said first fastening element (16) and a second end fixed to said second fastening element (17).

13. The implanting system (1) for implanting a totally implantable device (2) in a human or animal body according to claim 12, characterised in that said spiral spring (19), in said fastening position, is tensioned by traction.

14. The implanting system (1) for implanting a totally implantable device (2) in a human or animal body according to claim 11, characterised in that said second fastening element (17) is formed by a longitudinal probe (20) slidably positioned inside said spiral spring (19).

15. The implanting system (1) for implanting a totally implantable device (2) in a human or animal body according to claim 11, characterised in that said device (2) has an electroneuromodulation and/or stimulation and/or diagnostic data recording module, an electrical energy accumulator and a longitudinal piezoelectric element (23) for generating electric pulses for the recovery of electrical energy from the natural movement of the tissue in which said device (2) is implanted, said longitudinal piezoelectric element (23) being retained by said clamp (15).

16. An implanting system for implanting a totally implantable device in a human or animal body comprising an endoscope equipped with at least one operative channel wherein it comprises a transparent cover element that is detachably attached to a distal end of said endoscope, said cover element having a rounded configuration that is outwardly convex, said cover element facilitating the sliding of said introducing device.
